(19) United States
(12) Patent Application Publication
(10) Pub. No.: US 2005/0009770 A1
(43) Pub. Date: Jan. 13, 2005

(54) Use of excipients to increase DNA uptake by swine muscle cells

(76) Inventors: Lynn D. Nelson, Paw Paw, MI (US); Paula J. Gaynor, New Palestine, IN (US); Rajendra Krishnan, Portage, MI (US)

Correspondence Address: SCULLY SCOTT MURPHY & PRESSER, PC, 400 GARDEN CITY PLAZA, GARDEN CITY, NY, 11530, US

(21) Appl. No.: 10/672,592

(22) Filed: Sep. 26, 2003

Related U.S. Application Data
(60) Provisional application No. 60/413,722, filed on Sep. 26, 2002.

(51) Int. Cl. A61K 48/00; A61K 31/715; A61K 39/08

(52) U.S. Cl. 514/44; 514/54; 424/239.1

(57) ABSTRACT

The present invention provides novel excipient formulations for delivering nucleic acids, particularly naked DNA, across cell membranes in vivo. The present invention also provides penetration enhancers which can be used to enhance delivery and uptake by cells of a wide variety of nucleic acids particularly therapeutic agents or DNA molecule containing a gene encoding a protein which can benefit normal animals, especially in the application of gene therapy and augmenting viability or survival rate of newborn farm animals.

### FIELD OF THE INVENTION

The present invention relates to the use of excipients to increase DNA uptake by swine muscle cells.

### BACKGROUND OF THE INVENTION

The therapeutic use of proteins in the treatment of diseases is limited by the need for repeated protein administration and the high cost of the protein. However, plasmids containing genes of interest may be delivered to tissues which serve as sites for synthesis and secretion of proteins that have effects elsewhere in the body. Skeletal muscle is a useful target to evaluate this approach because of its large mass, vascularity and accessibility (Blau & Springer, New England J of Medicine, 333(23) 1975). Since muscle fibers are nondividing, effective gene delivery could result in long term protein production. However, in some instances muscle cells do not readily take up the plasmid DNA. To overcome this obstacle, some researchers have experimented with various buffers (Hartikka et. al., Gene Therapy, 7, 2000), while others have investigated the use of a combination of poloxamers to enhance DNA uptake by muscle cells in vivo (Lemieux, et. al., Gene Therapy, 7, 2000).

The buffers and combined poloxamers of Hartikka, et al. and Lemieux, et al. were evaluated in mice. When the buffer that Hartikka, et al. found most effective in mice was tested in swine, there was no increase of DNA uptake or expression in response to the DNA injection. Similarly, poloxamers had no effect on DNA expression in swine muscle cells either. Lemieux et al. WO 00/47186 evaluated several specific combinations of poloxamers in swine. Like the buffers of Hartikka, et al., the poloxamers of Lemieux et al. (WO 00/47186) had no effect on DNA expression in swine muscle cells either.

Electroporation studies proved that plasmids containing the genes for SEAP (human soluble embryonic alkaline phosphatase), β galactosidase, or porcine erythropoietin (EPO) are expressed in vivo in swine muscle cells. However, direct injection of the plasmid DNAs into swine muscle cells, without electroporation, resulted in minimal responses for SEAP or β galactosidase, and no response for porcine EPO. This lack of response was attributed to the inability of DNA to penetrate the cell membrane.

With electroporation not considered a commercially viable technique at this time, the present invention was initiated to identify excipients that can enhance DNA uptake by swine muscle cells in vivo. Excipients of various chemical classes were tested in the present invention. The present invention describes excipients that increase plasmid DNA uptake, and subsequent expression, by swine muscle cells in vivo.

### SUMMARY OF THE INVENTION

The present invention provides novel formulations for delivering polynucleotides across cell membranes in vivo.

In one embodiment, the invention provides excipients or “penetration enhancers”, which can be combined with naked or free nucleic acids, such as DNA, to facilitate or enhance the ability of these nucleic acids to traverse cellular membranes, i.e. to increase uptake of these nucleic acids by cells, e.g., swine muscle cells.

The suitable penetration enhancers provided by present invention include, for example, surfactants, bacterial toxins, polysaccharides and other penetration enhancers.

The formulations of the present invention can be used to enhance delivery of a wide variety of therapeutic agents or other molecules, and enhance the uptake of the therapeutic agents or other molecules by cells, particularly in the application of gene therapy and improving viability or survival rate of newborn farm animals.

In one embodiment, the invention pertains to a method for enhancing expression of a nucleic acid in a cell by contacting the cell with at least one nucleic acid and at least one penetration enhancer, such that the expression of the nucleic acid is enhanced.

In another embodiment, the invention pertains to a method for treating a subject by administering an effective amount of at least one penetration enhancer and a nucleic acid of the present invention.

The penetration enhancer is administered concurrently with the nucleic acid.

### BRIEF DESCRIPTION OF THE DRAWINGS

FIG. 1 is a photograph that depicts one plus (+) X-gal staining of swine muscle tissue.

FIG. 2 is a photograph that depicts two plus (++) X-gal staining of swine muscle tissue.

FIG. 3 is a photograph that depicts three plus (+++) X-gal staining of swine muscle tissue.

FIG. 4 is a photograph that depicts four plus (++++) X-gal staining of swine muscle tissue.

FIG. 5 is a photograph that depicts negative X-gal staining of swine muscle tissue.

### DETAILED DESCRIPTION OF THE INVENTION

The present invention provides novel formulations for delivering polynucleotides, especially naked DNA, across the membrane of cells, especially swine muscle cells, in vivo.

By “naked DNA” is meant that the DNA was not previously polyplexed with other chemical moieties. By “polyplex” is meant molecular complexes containing a compound, such as DNA, associated with one or more co-polymer domains. The co-polymer domain functions as a “delivery enhancer” to facilitate delivery of the compound.

One embodiment of the present invention provides a method for increasing the uptake of nucleic acids, e.g., DNA, particularly naked DNA, by animal cells, by administering excipients and the nucleic acids simultaneously, to these cells. The preferred cells of the present invention are swine muscle cells. Swine muscle is a useful target because of its large mass, vascularity and accessibility. Since muscle fibers are nondividing, effective gene delivery could result in long term protein production.

By “excipients” or “penetration enhancers” is meant formulants or reagents that enhance or increase delivery of agents, such as therapeutic agents e.g. nucleic acids, across cellular membranes. Preferred excipients are selected from various chemical classes including surfactants, bacterial toxins, polysaccharides and other penetration enhancers as described hereinbelow.

Surfactants, or detergents, are chemical compounds that reduce the surface tension of an aqueous solution and allow the molecules in solution to more efficiently come into contact with surrounding materials, thereby facilitating for enhanced uptake by these materials. In one embodiment the molecules are the plasmid DNA and the surrounding materials are the cell membranes. Surfactants provided by the present invention include, but are not limited to Triton X-100, sodium dodecyl sulfate, Pluronics (F68, P65, P84, F127, 25R2, and L62), Tween 20, and Tween 80, preferably, Tween 80, more preferably, Tween 80 at a concentration of 0.03-0.07%.

In accordance with the present invention bacterial toxins facilitate uptake of plasmid DNA by cells by causing temporary damage to the cell membrane through which the plasmid DNA can enter the cell. Examples of suitable bacterial toxins contemplated by the present invention include, but are not limited to, streptolysin O, cholera toxin, and recombinant modified labile toxin (mLT, Tulane University) of E. coli, preferably, E. coli rmLT, more preferably, E. coli rmLT at a dosage of 23-27 ug.

Aqueous solutions of polysaccharides can disrupt the osmotic pressure in the vicinity of the cell membrane, allowing for efficient movement of the plasmid DNA across the cell membrane. Suitable polysaccharides provided by the present invention include, but are not limited to, glucose, sucrose, fructose, trehalose, and maltose, preferably, sucrose, more preferably, sucrose at a concentration of 3-7%.

Examples of the penetration enhancers of the present invention include, but are not limited to, dimethyl sulfoxide (DMSO) and SEPA. DMSO is a penetrating solvent that enhances absorption of therapeutic agents through the skin. SEPA solution is another suitable penetration enhancer for use in the present invention. SEPA solution is also known by these designations (1,3-Dioxolane, 2-nonyl-(6CI, 7CI, 8CI, 9CI), 2-(1-Nonyl)-1,3-dioolane; 2-n-Nonyl-1,3-dioxolane; 2-Nonyldioxolane; Decanal ethylene acetal; Decanal, cyclic 1,2-ethanediyl acetal; SEPA 009; SEPA-I) and has the formula C₁₂H₂₄O₂, with the following structure. Preferred penetration enhancer of the present invention is DMSO, more preferably, DMSO at a concentration of 18-22%.

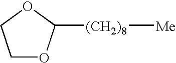

In accordance with the present invention, the excipients that increase plasmid DNA uptake also increase subsequent expression in swine muscle cells in vivo.

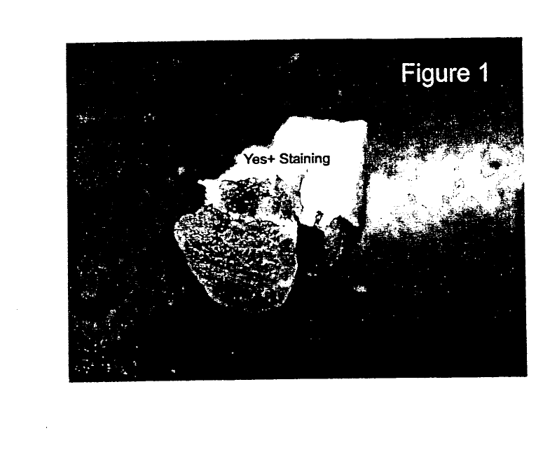

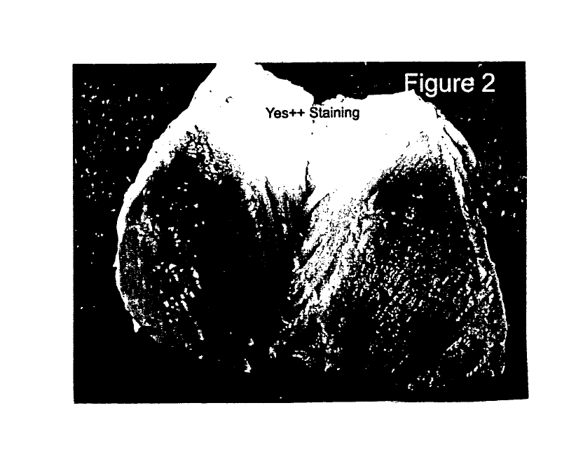

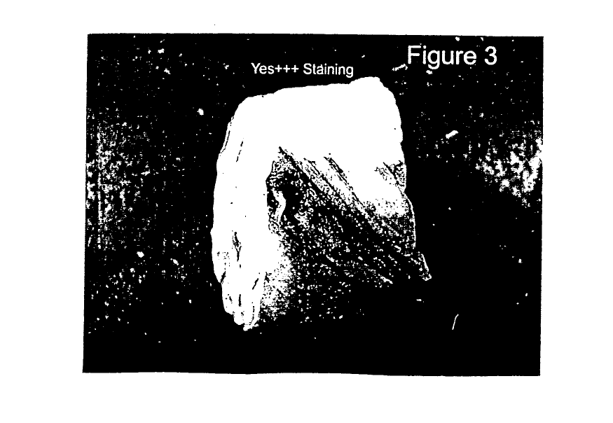

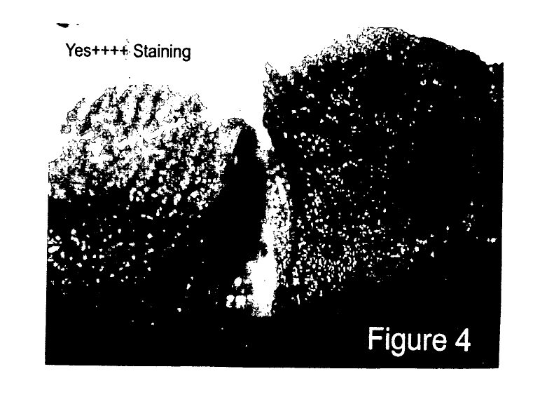

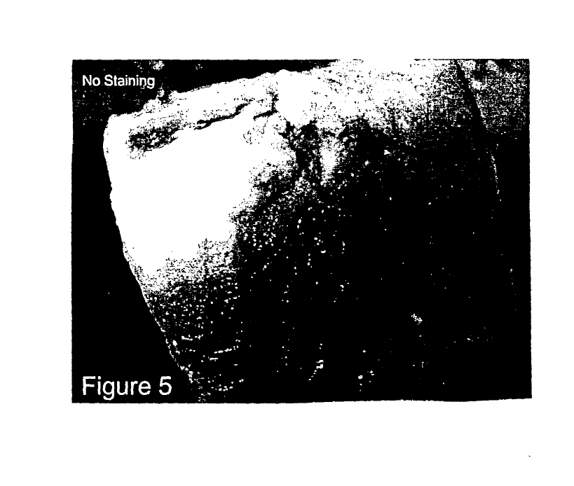

In one embodiment, the present invention relates to a method for enhancing expression of a nucleic acid, particularly naked DNA, in a cell. The method includes administering compositions of the invention, e.g. a solution of DNA and excipients to a subject, e.g. a pig, such that with the assistance of penetration enhancers, the nucleic acid traverses into the cell and expression of the nucleic acid is enhanced. Table 1 and FIGS. 1-5 exemplify the results of methods for enhancing expression of nucleic acids.

The composition of the present invention can be administered in vivo by intramuscular injection. The compositions are preferably injected intramuscularly in the form of a solution. Appropriate dosages may be determined empirically, as is routinely practiced in the art. However, it is contemplated that a dosage of about 3-7% sucrose, 18-22% DMSO, 0.03-0.07% Tween 80, or 23-27 μg rmLT can be used to increase DNA uptake by swine muscle cells.

By “enhanced” is meant any expression of a nucleic acid, for example, but not limited to, a plasmid containing the genes encoding human soluble embryonic alkaline phosphatase (SEAP), β galactosidase, or porcine erythropoietin (EPO), that is greater than that observed by administering the nucleic acid to a subject or a culture of cells without any penetration assistance of the excipients.

The term “subject” includes organisms and cells including protists, birds, reptiles, monera, bacteria, and preferably, mammals, especially, pigs.

By “treat”, “treating” or “treatment” is meant that at least one symptom associated or caused by the state, disorder or disease is diminished or alleviated, or at least one benefit unexpected under the normal condition, is achieved. For example, treatment can include diminishment of one or several symptoms of a disorder or complete eradication of a disorder in a subject compared with a subject without treatment. Treatment, in accordance with the present invention is also directed to or providing a benefit to farmers by augmenting the survival rate or viability of normal newborn farm animals compared with the survival rate of newborn farm animals without treating pregnant mothers.

In another embodiment, the present invention relates to a method for treating a subject suffering from a genetic or an acquired disorder by administering an effective amount of at least one penetration enhancer and a nucleic acid of the present invention to ensure the enhanced expression of the nucleic acid and thereby the subject's disorder or symptom is diminished or eradicated.

In one embodiment, a subject is a normal subject. By “normal subject” is meant an animal not suffering from a genetic or an acquired disorder.

In another embodiment, the present invention relates to a method for treating a subject and for example, an animal subject, by administering an effective amount of at least one penetration enhancer and a nucleic acid of the present invention to ensure the enhanced expression of the nucleic acid and thereby a benefit or better result unexpected under normal conditions, is achieved. By “normal condition” is meant that no treatment is administered. For example and in accordance with the present invention, a plasmid containing the porcine EPO-encoding gene can be administered with excipients to a normal pregnant pig so that the blood cell level in mother pig is increased. It is contemplated by the present invention that increased blood cell numbers in mother pig will result in increased oxygenation, thereby resulting in an augmented survival rate or viability of piglets.

The penetration enhancers or the excipients should be administered concurrently with the nucleic acid.

Formulants used to enhance delivery and uptake of a wide variety of therapeutic agents and other molecules by cells, particularly in application of gene therapy and viability augmentation, are also provided by the present invention.

This invention will now be further illustrated by the following non-limiting example.

### EXAMPLES

### DNA/excipient Preparations

Solutions of sucrose (3-7%), DMSO (18-22%), Tween 80 (0.03-0.07%), or E. coli recombinant modified labile toxin (rmLT) (23-27 ug/dose) can be formulated at the desired final concentration in 150 mM sodium phosphate, pH 7.2, containing 1 mg/ml of the plasmid DNA of interest. Solutions can be assembled by dissolving the excipients and plasmid DNA in 150 mM sodium phosphate, pH 7.2 at the desired concentration. Alternatively, the preparations can be prepared as sub-solution, at twice their final concentration in 150 mM sodium phosphate, pH 7.2, and then mixed in equal volumes immediately prior to administration. The SEPA-DNA solution is prepared by adding 1 part of the respective DNA solution, at 5 mg/ml, to 4 parts SEPA.

Table 1 contains the scoring results of X-gal staining in swine muscles 5 days following injection of the various DNA and excipients solutions.

### Excipient Testing in Pigs

Each pig received a total of 6 injections. All injections administered in the longissimus dorsi muscle (3/side). Injections were approximately 2.5″ lateral to the spine. On each side of the pig, one site was located at approximately the last rib, and the two others were at approximately 6″ and 12″ cranial (towards the nose) from the last rib. Hair was clipped in an area approximately 1″ square to aid identification of injection sites. Injections were made in the center of each shaved area, at approximately 90′ angle to the skin surface. The pigs were restrained briefly in a self-locking head catch chute and the test article administered IM using a 1.5″ long 18 gauge needle attached to a 3 ml syringe. Each test article (treatment) was administered in a total of 3 injection sites (each on a different pig). The total volume of each injection was 1 ml.

Pigs were euthanized 5 days following administration of the respective DNA and excipient solution. A section of tissue approximately 1″ cubed, surrounding the point of entry of the needle, was excised. These tissue sections were sliced 90% through, in sections approximately 2-3 mm wide, beginning at the edge closest to the skin. The sliced tissue sections were placed in a sufficient volume of 4% paraformaldehyde to completely cover the tissue and incubated at 4° C. for 3 hours. Following the 3 hour incubation period the fixed tissue sections were rinsed 3 times with PBS. The rinsed tissues were then stained with a solution of 40 μM MgCL₂, 0.5 mM ferric-ferrocyanide, 0.05% deoxycholate and 0.54 mg/ml 5-bromo-4chloro-3indoyl-β-D-galactoside (X-gal) for 18 hours at 37° C. After straining was complete, the tissues were washed 3 times with 3% DMSO in PBS. The amount of staining was determined by visual observation using a subjective scale from “No Staining” through a gradation to “Yes++++”. The criterion for grading was the intensity and amount of FIGS. 1-5).

TABLE 1

| Pig # | Treatment | β galactosidase staining |
|---|---|---|
| 132 | Negative control (150 mM sodium | No staining |
| | phoshate) | |
| 133 | Negative control (150 mM sodium | No Staining |
| | phosphate) | |
| 134 | Negative control (150 mM sodium | No Staining |
| | phosphate) | |
| 135 | 5% Sucrose | No Staining |
| 136 | 5% Sucrose | Yes++ |
| 137 | 5% Sucrose | Yes+++ |
| 132 | 20% Dimethyl sulfoxide | Yes+++ |
| 133 | 20% Dimethyl sulfoxide | Yes+ |
| 134 | 20% Dimethyl sulfoxide | Yes++ |
| 117 | 0.05% Tween80 | Yes++ |
| 118 | 0.05% Tween80 | Yes++ |
| 119 | 0.05% Tween80 | Yes+++ |
| 144 | 25 ug rmLT/dose | Yes++ |
| 145 | 25 ug rmLT/dose | Yes+++ |
| 146 | 25 ug rmLT/dose | Yes++ |

### Conclusion

A combination of either 5% Sucrose, 20% Dimethyl sulfoxide, 0.05% Tween 80 and 25 ug rmLT/dose, or SEPA solution with plasmid DNA (prepared by adding 1 part of the respective DNA solution, at 5 mg/ml, to 4 parts SEPA), containing a β galactosidase gene, resulted in enhanced DNA uptake by swine muscle cells, as indicated by tissue staining. Therefore, these excipients can specifically increase DNA uptake, and the resulting protein, in vivo, in swine muscle cells.

1. A method of increasing uptake of nucleic acids by swine muscle cells which comprises administering at least one excipient and at least one nucleic acid to a swine muscle cell wherein such excipient enhances the ability of the nucleic acid to traverse swine muscle cell membranes.

2. A method of enhancing expression of a nucleic acid in a swine muscle cell, comprising administering a composition comprising at least one excipient and at least one nucleic acid to a swine muscle cell.

3. A method of treating a subject comprising administering an effective amount of at least one excipient and at least one nucleic acid to enhance expression of said nucleic acid in a subject.

4. The method of claims 1-3, wherein said excipient is selected from the group consisting of surfactants, bacterial toxins, and polysaccharides.

5. The method of claim 4, wherein said surfactant is selected from the group consisting of Triton X-100, sodium dodecyl sulfate, Pluronics (F68, P65, P84, F127, 25R2, and L62), Tween 20 and Tween 80.

6. The method of claim 5, wherein the surfactant is Tween 80.

7. The method of claim 6, wherein the concentration of said Tween 80 is 0.03-0.07%.

8. The method of claim 4, wherein said bacterial toxin is selected from the group consisting of streptolysin O, cholera toxin, and recombinant modified labile toxin (rmLT) of E. coli.

9. The method of claim 8, wherein said bacterial toxin is E. coli rmLT.

10. The method of claim 9, wherein the dosage of said E. coli rmLT is 23-27 ug.

11. The method of claim 4, wherein said polysaccharide is selected from the group consisting of glucose, sucrose, fructose, trehalose, and maltose.

12. The method of claim 11, wherein said polysaccharide is sucrose.

13. The method of claim 12, wherein the concentration of said sucrose is 3-7%.

14. The method of claim 4 further comprising dimethyl sulfoxide (DMSO) and SEPA.

15. The method of claim 14, wherein the excipient is DMSO.

16. The method of claim 15, wherein the concentration of said DMSO is 18-22%.

17. The method of claim 3, wherein said subject is a protist, a bird, a reptile, a monera, a bacterium, and a mammal.

18. The method of claim 17, wherein said mammal is a pig.

19. The method of claim 3, wherein said subject has a genetic or an acquired disorder which is diminished or eradicated after the treatment, compared with an untreated subject.

20. The method of claim 19, wherein said subject is a pig.

21. The method of claim 3, wherein said subject is a normal subject.

22. The method of claim 21, wherein said subject is a normal pregnant pig.

23. The method of claim 22, wherein the survival rate or viability of piglets is augmented by treating said normal pregnant mother pig with a plasmid containing a gene encoding porcine erythropoietin together with an excipient.

24. The method of claim 2, wherein said composition is administered by intramuscular injection.

25. The method of claims 1-3, wherein said excipients are administered concurrently with the nucleic acids or the therapeutic agents.

26. The method of claims 1-3, wherein said nucleic acid is a DNA.

27. The method of claim 26, wherein said DNA is a naked DNA.

28. The method of claim 18, wherein said naked DNA is a naked plasmid DNA.
